(19) United States
(12) Patent Application Publication
(10) Pub. No.: US 2021/0089043 A1
(43) Pub. Date: Mar. 25, 2021

(54) SYSTEM FOR COOPERATIVE MOVEMENT CONTROL AND/OR MOVEMENT SUPERVISION OF MOBILE MEDICAL COMPONENTS

(71) Applicant: Siemens Healthcare GmbH, Erlangen (DE)

(72) Inventor: Andreas DEINLEIN, Bayreuth (DE)

(73) Assignee: Siemens Healthcare GmbH, Erlangen (DE)

(21) Appl. No.: 17/020,938

(22) Filed: Sep. 15, 2020

(30) Foreign Application Priority Data
Sep. 23, 2019 (EP) 19198939.1

(51) Int. Cl. G05D 1/02; G01C 21/20; B60K 1/04; G16H 40/63; G06N 20/00

(52) U.S. Cl. CPC G05D 1/0221; G01C 21/206; G05D 1/0287; A61B 6/4405; B60K 1/04; G16H 40/63; G06N 20/00; G05D 1/0257

(57) ABSTRACT

In one embodiment, the invention relates to a system for cooperative movement control and/or movement supervision of mobile medical components in a medical facility. The medical facility, such as e.g. a hospital, has a plurality of facility units, comprising a plurality of mobile medical components, which are connected to each other via a communication link and communicate continuously, in order via a communication protocol cooperatively to supervise and/or control the movement of each mobile medical component of the plurality of mobile medical components in the medical facility and/or in the facility units.

### PRIORITY STATEMENT

The present application hereby claims priority under 35 U.S.C. § 119 to European patent application number EP 19198939.1 filed Sep. 23, 2019, the entire contents of which are hereby incorporated herein by reference.

### FIELD

Embodiments of the invention generally relate to a system for cooperative movement supervision and/or movement control of mobile medical components in a medical facility with a plurality of facility units. Embodiments of the invention further generally relate to a corresponding mobile medical component, to a method for operating the system and to a method for operating the medical component, and also to a computer program.

### BACKGROUND

In a medical facility, for example in a hospital, medical components (e.g. imaging devices) from different manufacturers are provided for different purposes, such as e.g. for a medical diagnosis. The medical components are specified, developed and constructed independently of one another. In most cases said components involve proprietary specific solutions. These proprietary specific solutions can have communication methods, communication interfaces and/or data processing that are not compatible with other devices and thus do not make communication or exchange of data between the different medical components possible. The disadvantage here is that these medical components cannot cooperate with one another in order to make possible a common sequence of operations in a hospital. This restricts the opportunity for automation in a medical facility and thereby prevents an efficient workflow in the medical facility.

Solutions envision the use of specifically embodied interfaces, in which for example an angiography device can exchange data with a computed tomography device. The requirement for this is that a medical component, for example the angiography device, is the master and implements the necessary interface for communication. This, however, represents a specific interface, via which only these two aforementioned medical components can communicate and exchange data and thus together realize a specific tailored clinical workflow. This communication, provided specifically from one device to another device, does not however make it possible to accept further participating devices, in order thereby to set up and provide an holistic clinical workflow.

The absence of or restriction to communication and the absence of data exchanged becomes even more problematic when medical components which can be driven by motors (medical AGVs—Automated Guided Vehicles) are used in a medical facility. The medical driverless transport vehicles are floor-based medical components with their own drive units. On request these can be automatically controlled and driven without making contact with each other. In order to make possible an efficient deployment and sequence and without collisions and/or disruptions to the medical AGVs, agreement and in particular cooperation between the medical AGVs within the medical facility is necessary.

### SUMMARY

The inventors have discovered that a need exists for a mechanism for cooperative movement supervision and/or movement control of mobile medical components in a medical facility with a plurality of facility units. At least one embodiment of the present invention sets out to achieve is to create a solution that at least partly overcomes at least one of the known disadvantages in the prior art.

Embodiments are directed to a system, a mobile medical component, a method and a computer program.

In accordance with a first embodiment, the invention relates to a system for cooperative movement supervision and/or movement control of mobile medical components (e.g. drivable medical devices, e.g. imaging devices) in a medical facility. The medical facility, such as a clinic for example, is divided or structured for the system into a number of facility units, such as e.g. rooms, areas (operating theatre, sterile area). The system comprises and controls a plurality of mobile medical components, which exchange data with one another via a communication link and communicate continuously, in order, via a communication protocol, cooperatively to monitor and/or to control the movement of each mobile medical component of the plurality of mobile medical components in the medical facility and/or in the facility units.

In the sense of an embodiment of the present invention, a medical facility comprises a facility in which medical examinations and/or treatments are carried out using medical components. The medical facility can be a hospital, an ambulance, a medical research laboratory, a hospital ward and/or a polyclinic or be embodied as such. The list given by way of example here does not represent a conclusive listing or restriction. Instead further facilities are conceivable in which medical examinations and/or treatments can take place. The medical facility can comprise a plurality of facility units. A facility unit can be a room as a delimited area, a corridor, a staircase, an elevator as a movement area and/or a non-delimited area (free area).

Furthermore, in the sense of an embodiment of the present invention, a mobile medical component is to be understood as a component that has its own drive and its own power supply. The mobile medical component can be a mobile couch on which patients lie for the examination for example. Moreover a mobile medical component can be an examination device, a device for carrying out an imaging method, for example CT, MRT, x-ray or ultrasound. Medical components also further comprise medical or clinical devices that are used for diagnosis, recovery and/or life support.

In accordance with a second embodiment, the invention relates to a mobile medical component from a quantity of mobile medical components for cooperative movement supervision and/or movement control of the quantity of mobile medical components. The mobile medical component comprises a drive unit, which is embodied to drive the mobile medical component. The drive unit comprises a DC motor and can include a transmission.

The mobile medical component further comprises a power supply, which is embodied for electrical supply of the individual components of the mobile medical component. The power supply can be charged via a DC voltage source or via an AC voltage source in conjunction with an on-board charger in the mobile medical component or a rectifier in the AC voltage source.

The mobile medical component comprises a communication interface, which is embodied to set up a communication link with further mobile medical components in a medical facility. The communication interface is embodied to set up a communication link via WLAN, Bluetooth, Bluetooth Low Energy or infrared.

Furthermore the mobile medical component comprises a sensor unit for detecting the environment of the mobile medical component. In a possible form of embodiment the sensor unit comprises at least a lidar system or a ladar system. A lidar (light detection and ranging) or ladar (Laser detection and ranging) system is a measuring method that measures the distance to then target by illuminating the target with pulsed light, e.g. laser light, and measures the reflected pulses with a sensor. Differences in the laser return times and/or the wavelength can also be used to create the digital 3D representations of the target.

In accordance with a third embodiment, the invention relates to a method for operating a system as described here by providing control signals created locally and autonomously on the medical component for cooperative movement supervision and/or movement control of the quantity of mobile medical components in a facility and/or in a plurality of facility units of the facility.

In accordance with a fourth embodiment, the invention relates to method for operating a mobile medical component of a quantity of mobile medical components in a medical facility comprising a drive unit, a power supply, a communication interface, a sensor unit, a memory unit and a processor unit. The method comprises the following steps:

Activation of the communication interface for continuously receiving navigation signals from each of the mobile medical components of the quantity of mobile medical components in the medical facility;

Receipt of navigation signals from each of the mobile medical components;

Creation of control signals for a drive unit for movement of the mobile medical component based upon the received navigation signals and/or a movement model and/or a request signal;

Creation of navigation signals via a communication protocol based upon the control signals created; and

Sending of the created navigation signals via the communication interface for cooperative supervision and/or control of each mobile medical component of the plurality of mobile medical components in the medical facility and/or in the facility units.

The inventive form of embodiment of the method described here in accordance with the third embodiment of the invention can also be embodied as a computer program, wherein a computer is made to carry out an embodiment of the inventive method described above when the computer program is executed on a processor of the computer. The computer program can be provided as a signal by download or stored in a memory unit of the computer or of the mobile medical component with computer-readable program code stored therein, to make the mobile medical component execute the instructions in accordance with the method stated above. In this case the computer program can also be stored on a machine-readable storage medium. An alternate solution makes provision for a storage medium that is intended for storage of the computer-implemented method described here and is able to be read by a computer or processor.

At least one embodiment is directed to a system for at least one of cooperative movement supervision and movement control of mobile medical components in a medical facility including a plurality of facility units, comprising:

a plurality of mobile medical components, to exchange data via a communication link and communicate continuously, and to, via a communication protocol, at least one of cooperatively supervise and control the movement of each of the mobile medical components of the plurality of mobile medical components, at least one of in the medical facility and in the plurality of facility units.

At least one embodiment is directed to a mobile medical component including a quantity of mobile medical components for at least one of cooperative movement supervision and cooperative movement control of the quantity of mobile medical components, comprising:

a driver, embodied to drive the mobile medical component;

a power supply, embodied to supply electrical power to components of the mobile medical component;

a communication interface, embodied to set up a communication link to further mobile medical components in a medical facility;

a sensor to detect an environment of the mobile medical component;

a memory to store at least one of a learned movement model and maps of the medical facility; and

a processor for at least one of cooperative supervision and cooperative control of movement of each mobile medical component in at least one of the medical facility and facility units, through provision of control signals for activating the drive unit.

At least one embodiment is directed to a method for operating a system including a plurality of mobile medical components, to exchange data via a communication link and communicate continuously, and to, via a communication protocol, at least one of cooperatively supervise and control the movement of each of the mobile medical components of the plurality of mobile medical components, at least one of in the medical facility and in the plurality of facility units, comprising:

provisioning control signals created locally and autonomously on the medical component for at least one of cooperative movement supervision and cooperative movement control of the quantity of mobile medical components in at least one of a facility and a plurality of facility units of the facility.

At least one embodiment is directed to a method for operating a mobile medical component of a quantity of mobile medical components in a medical facility including a driver, a power supply, a communication interface, a sensor, a memory and a processor, the method comprising:

activating the communication interface for continuous receipt of navigation signals from each of the mobile medical components of the quantity of mobile medical components in the medical facility;

receiving navigation signals from each of the mobile medical components;

creating control signals for the driver for movement of the mobile medical component based upon at least one of the navigation signals received, a movement model and a request signal;

creating navigation signals via a communication protocol based upon the control signals created; and

sending the navigation signals created, via the communication interface, for at least one of cooperative supervision and cooperative controlling each mobile medical component of the plurality of mobile medical components in at least one of the medical facility and the facility units.

At least one embodiment is directed to a non-transitory computer readable medium storing a computer program including program code for carrying out the method of at least onew embodiment when the computer program is executed on an electronic device.

### BRIEF DESCRIPTION OF THE DRAWINGS

The present invention will be explained in greater detail below with reference to example embodiments specified in the schematic figures of the drawings. In the figures:

FIG. 1 shows a schematic diagram of a form of embodiment of a system for cooperative movement supervision and/or movement control;

FIG. 2 shows a block diagram of a communication layer model for communication between the mobile medical components;

FIG. 3 shows a schematic diagram of a time sequence of the registration of a new mobile medical component;

FIG. 4 shows a schematic diagram of a time sequence of the registration of data communication between the mobile medical components;

FIG. 5 shows a schematic diagram of a time sequence of the registration of the movement request of a mobile medical component;

FIG. 6 shows a schematic diagram of a time sequence of the registration of a master request of a mobile medical component;

FIG. 7 shows a further block diagram in accordance with a form of embodiment of the mobile medical components;

FIG. 8 shows a flow diagram in accordance with a form of embodiment of the inventive method.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. The present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (procesor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

In accordance with a first embodiment, the invention relates to a system for cooperative movement supervision and/or movement control of mobile medical components (e.g. drivable medical devices, e.g. imaging devices) in a medical facility. The medical facility, such as a clinic for example, is divided or structured for the system into a number of facility units, such as e.g. rooms, areas (operating theatre, sterile area). The system comprises and controls a plurality of mobile medical components, which exchange data with one another via a communication link and communicate continuously, in order, via a communication protocol, cooperatively to monitor and/or to control the movement of each mobile medical component of the plurality of mobile medical components in the medical facility and/or in the facility units.

In an advantageous way, the movement of each mobile medical component is cooperatively monitored and/or controlled by an embodiment of the present invention. In the sense of an embodiment of the present invention, cooperative monitoring and/or control is to be understood as an interactive and/or a collaborative monitoring and/or control, so each of the plurality of mobile medical components is embodied for monitoring and control.

Each mobile medical component of the plurality of mobile medical components has its own processor unit and thus its own local intelligence, via which there can be a monitoring and/or control of the mobile medical components. Each mobile medical component can receive navigation signals of the further mobile medical components and via its own processor unit (local intelligence) and create and provide navigation signals.

The created navigation signals can be provided for control of the mobile medical component creating the navigation signal and/or for the further mobile medical components. Thus an autonomous movement of a number of mobile medical components in a medical facility, in particular in an automated medical facility without a central intelligence is made possible.

In particular there can be a common movement supervision and/or movement control. The movement supervision and/or movement control is executed in a facility-specific and preferably facility unit-specific way. This means that the movement control and/or supervision of the respective mobile medical component is undertaken as a function of the facility unit in which the component is located (ACTUAL position) or to which it should move (requested NOMINAL position). If for example a first facility unit includes a prohibited zone and a second facility unit does not include any prohibited zone, the movement control for the respective component is undertaken in the first facility unit other than and differently from the second facility unit, namely while taking into consideration the prohibited zone.

It is further advantageous that mobile medical components that do not include the necessary sensor unit for detection of the route or a map can receive via the continuous communication link an update about changes in the facility units or to the map. This information can be created and provided by other mobile medical components with the corresponding sensor unit.

In accordance with an embodiment of the invention, the mobile medical components are connected to each other via at least one communication link. In accordance with a preferred form of embodiment of the present invention the communication link is established via a Wi-Fi connection (WLAN). Wi-Fi is a technology for wireless local networking of devices, for example mobile medical components, which are based on the IEEE 802.11 standard. A Wi-Fi connection makes possible a wireless connection of a device and/or a mobile medical component to a communication gateway with a high bandwidth of 72 to 9608 Mbit/s, depending on the version (Wi-Fi 4, Wi-Fi 5, Wi-Fi 5) of the standard used. Therefore data can be transmitted from the mobile medical component to the communication gateway and to the further mobile medical components.

In accordance with a further preferred form of embodiment of the present invention, the communication link is established by a Bluetooth connection. The Bluetooth connection is a wireless technology standard for the exchange of data between the communication gateway and the mobile medical component over a short distance using shortwave UHF radio waves of 2.400 to 2.485 GHz. In this way data can be transmitted from the mobile medical component to the communication gateway and to the further mobile medical components.

In accordance with a further form of embodiment of the present invention, the communication link is established by a Bluetooth Low Energy (BLE) connection. Bluetooth Low Energy is a wireless Personal Area Network technology that can be used to link mobile devices in an area of around 10 meters. Bluetooth Low Energy has a far lower power consumption and lower costs with the same range. In this way data can be transmitted from the mobile medical component to the communication gateway and to the further mobile medical components.

In accordance with a further form of embodiment of the present invention, the communication link can be established via an infrared connection. An infrared connection makes possible wireless data transmission for a maximum range over the last meter according to the point-and-shoot principle. Infrared communication offers a physically safer data transmission with a line of sight and has a very low bit error rate, which makes it very efficient. Via an infrared connection, data can be transmitted directly from the one mobile medical component directly to the further mobile medical components in the medical facility when these are in a line of sight and/or are passing one another.

Via the communication links there is a continuous communication between the mobile medical components in the medical facility. Here the mobile medical components are embodied depending on their function and use (master or slave) in such a way that, via the communication link, there is a constant listening for receiving (slave) of new navigation signals and/or data. A mobile medical component embodied as master can constantly send navigation signals and/or data.

In one form of embodiment, the continuous communication can comprise a periodic continuous communication. Thus the bandwidth used and data load can be reduced.

In accordance with a form of embodiment of the invention, the communication protocol of the mobile medical components is embodied as a master-slave protocol. Here one mobile medical component is chosen as the master and the further mobile medical components are chosen as slaves. In an advantageous way there is thus a handshake between the medical components involved, in which the status of the master is transferred to another medical component. Only the medical component that has the status of the master may remotely control other medical components involved in the current workflow. This enables the cooperation and control as well as the workflow of the medical components to be optimized for availability.

In one form of embodiment the communication between the medical components can be undertaken via a publish-subscribe protocol (DDS) in accordance with the Robot Operating system (ROS2). Here each component can communicate with any other without the need existing for a “master” for control of the communication. In an advantageous manner the communication protocol is also capable of functioning on failure of one or more medical components. A failure here can also include the master. This would even continue to allow the addition of further medical components via plug&play for example.

The mobile medical components necessary for an examination can be selected via a request signal at a user selection interface by a user, for example a doctor performing the treatment and defined as so-called master components. The status as master can be defined for the time of the examination and the further mobile medical components necessary for the examination are assigned the status of slave. The status can be stored and/or transmitted in a status message. The status message can comprise further information about the mobile medical component, such as e.g. its current position, which displays information as to whether it has been requested, whether it has been permitted for an enabling signal, how frequently it is requested etc.

Each mobile medical component can display the status assigned to it in each case in the status message via a user interface, for example via a display. The master component can provide navigation signals in order to remotely control the slave components. In an advantageous manner there can thus be an efficient and optimized movement and provision of the mobile medical components. Moreover the supervision and control takes place in such a way that no collision between the mobile medical components occurs.

In a further form of embodiment, the role of the master can be given by one mobile medical component to another mobile medical component. This is of advantage if, during an examination and/or treatment, there is a change of the medical component. The safety relating to the movement of the mobile medical components is enhanced by this.

In accordance with a further form of embodiment of the invention, a facility unit of the plurality of facility units of the medical facility comprises at least two separate task-specific zones. A facility unit, for example a treatment room, comprises at least separate two task-specific zones. The task-specific zones represent defined areas. In these areas only the tasks allocated to said areas may be carried out. The task-specific zones can be defined via coordinates, markers and/or boundary markings. The coordinates, markers and/or boundary markings can be detected via a sensor unit. In one form of embodiment the coordinates, markers and/or boundary markings can be learned in a movement model and/or stored in a map.

In accordance with a further form of embodiment of the invention, one of the task-specific zones comprises a prohibited area. A prohibited area represents a zone that may not be entered by any mobile medical component. In this way it is ensured that no mobile medical component moves into the prohibited area in which other objects may possibly be located (e.g. a patient) that would be disturbed or injured or rendered incapable of functioning by the entry of a mobile medical component.

In accordance with a further form of embodiment of the invention, one of the task-specific zones comprises a transfer area. The transfer area represents a zone via which there is the movement and/or the transfer of a mobile medical component between the autonomy area and the movement area or between the waiting area and the movement area. It is thus ensured that no mobile medical component moves into movement area without an enabling signal and causes a collision.

In accordance with a further form of embodiment of the invention, one of the task-specific zones comprises a movement area. The movement area represents a zone, in which a mobile medical component may move after receiving an enabling signal. It is ensured via the enabling signal that no mobile medical component enters this zone without permission. Any danger to persons in this area is thus minimized. The examination and/or treatment can be carried out in the movement area for example.

In accordance with a further form of embodiment of the invention, one of the task-specific zones comprises a waiting area. The waiting area represents a zone in which a mobile medical component can be positioned in a wait state. Collisions between a waiting mobile medical component and a moving medical component can thus be avoided.

In accordance with a further form of embodiment of the invention, one of the task-specific zones comprises an autonomy area. The autonomy area represents a zone in which the mobile medical components can move autonomously and without an enabling signal.

In accordance with a further form of embodiment of the invention, the movement of a mobile medical component into the movement area is enabled via an enabling signal. The enabling signal can be provided via a user interface. The user interface is embodied to receive an input from a user. The user interface can be embodied via an electronic device, for example a computer or a handheld device. This makes possible an efficient and rapid choice of the corresponding mobile medical component. Moreover the status or the status message of the chosen mobile medical component can be output. The user interface communicates via the communication link with the mobile medical components.

In an alternate form of embodiment the enabling signal can be provided via a switch panel with switches and/or pushbuttons. The switch panel can be connoted to a computer and/or a control for creating the enabling signal. The computer and/or the control communicate via the communication link with the mobile medical components.

In accordance with a further form of embodiment of the invention, the mobile medical components move autonomously in the medical facility using a learned movement model. This has the advantage that the mobile medical components, after receiving a request signal, navigate autonomously to the facility unit in which the request signal was triggered. The request signal contains the information about the requested mobile medical component and the facility unit in which the mobile medical component is to be used. The request signal can be provided via the user interface for issuing the enabling signal.

In accordance with a further form of embodiment of the invention, the movement model is learned via a movement profile driven manually. The movement profile can be created for each of the different routes of the respective mobile medical component by a manual driving of the routes with the respective medical component. The respective manually driven movement profiles are learned in the movement model. In an advantageous manner no additional hardware is necessary for a localization.

In accordance with a further form of embodiment of the invention, the movement is learned via a predetermined movement profile. In an advantageous manner the route can be learned directly via a movement profile, whereby it does not have to be driven manually. The movement profile comprises navigation instructions for control and/or navigation of the mobile medical component in the medical facility. For example the movement profile can have navigation instructions about the direction of movement, speed of movement and/or change in direction of movement of a mobile medical component.

In accordance with a further form of embodiment of the invention, the mobile medical components orient and move themselves autonomously in the medical facility via a localization system. The localization system comprises coordinates of the medical facility. The coordinates describe points in the different facility units in the medical facility. The coordinates can form different routes. According to the mobile medical component and its possible uses different routes are produced with different coordinates. The coordinates can be established via an indoor position determination method. The indoor position determination can take place via WLAN, BLE, ultra wideband (UWB) and/or Radio-Frequency Identification (RFID). An existing WLAN network can be used for a WLAN positioning system (WPS). The WPS is a geolocalization system, which uses the characteristics of the adjacent WLAN access points and other wireless access points to find out where the mobile medical component is located. A well-established and widely used localization technique, which is used for positioning with wireless access points is based on measuring the intensity of the received signals. Typical parameters that are useful for the geolocalization of the wireless access points, are SSID and MAC address.

To determine the position via Bluetooth, Low Energy Bluetooth signals can be received from Bluetooth beacons installed in the medical facility. The position can be determined via a signal strength measurement.

The mobile scanning facility can receive Bluetooth signals from beacons, which are installed in the scan environment. The signal strength measurement can be used to determine the position of the beacons.

UWB is a short-range radio technology. The accuracy is at below 30 centimeters and is thus much greater than that of beacons or WLAN.

RFID comprises an automatic and contactless method for identification and localization with radio waves.

In accordance with a further form of embodiment of the invention, the mobile medical components move in the medical facility autonomously via an SLAM method. The SLAM method computationally solves the problem of creating a map (digital representation of the environment) of an unknown environment (medical facility) or updating it and simultaneously following the location of the mobile medical component on the map. The map is built up incrementally. Starting from the position of the mobile medical component as the starting point in the map, a representation of the environment can be built up and continuously followed. Thus the mobile medical component during the scan time (measuring of the environment) obtains ever better knowledge about the environment and the facility units in the medical facility. This knowledge is incrementally improved with each movement task. The data thus detected is used to provide control commands for the drive and steering system of the autonomous mobile medical component, which controls the autonomous mobile medical component along an application-specific scan route.

In accordance with a second embodiment, the invention relates to a mobile medical component from a quantity of mobile medical components for cooperative movement supervision and/or movement control of the quantity of mobile medical components. The mobile medical component comprises a drive unit, which is embodied to drive the mobile medical component. The drive unit comprises a DC motor and can include a transmission.

The mobile medical component further comprises a power supply, which is embodied for electrical supply of the individual components of the mobile medical component. The power supply can be charged via a DC voltage source or via an AC voltage source in conjunction with an on-board charger in the mobile medical component or a rectifier in the AC voltage source.

The mobile medical component comprises a communication interface, which is embodied to set up a communication link with further mobile medical components in a medical facility. The communication interface is embodied to set up a communication link via WLAN, Bluetooth, Bluetooth Low Energy or infrared.

Furthermore the mobile medical component comprises a sensor unit for detecting the environment of the mobile medical component. In a possible form of embodiment the sensor unit comprises at least a lidar system or a ladar system. A lidar (light detection and ranging) or ladar (Laser detection and ranging) system is a measuring method that measures the distance to then target by illuminating the target with pulsed light, e.g. laser light, and measures the reflected pulses with a sensor. Differences in the laser return times and/or the wavelength can also be used to create the digital 3D representations of the target.

In a further possible form of embodiment the sensor unit includes at least one camera system. The camera can be used for measuring distances, for differentiating and identifying objects in the environment and for provision of data for the localization of the mobile medical component.

In a further possible form of embodiment the sensor unit includes at least one radar system. The radar system can be used for measuring distances.

In a further possible form of embodiment, the sensor unit contains at least one acoustic system. Acoustic systems, such as e.g. ultrasound sensors, can be used for measuring distances and in order to provide data for the localization.

In a further possible form of embodiment, the sensor unit includes at least one infrared system. An infrared system can be used for measuring distances and in order to provide data for the localization.

The systems for measuring distances described above can be used individually or in combination in order to scan the scanning environment (i.e. the environment in which the mobile medical component is to move) and/or to detect objects or further mobile medical components in the scanning environment.

The mobile medical component of an embodiment furthermore comprises a memory unit for storing the learned movement model and/or maps of the medical facility. The memory unit can contain a read-only memory (ROM) and/or a random-access memory (RAM). The memory unit can also contain a hard disk drive for reading from and writing to a hard disk. The storage media offer a non-volatile storage of machine-readable instructions, data structures, program modules of the movement model and further data.

Moreover the mobile medical component of an embodiment comprises a processor unit (at least one processor) for cooperative supervision and/or control of the movement of each mobile medical component in the medical facility and/or in the facility units by the provision of control signals for activating the drive unit. The processor unit can also be embodied in an integrated circuit.

In accordance with a third embodiment, the invention relates to a method for operating a system as described here by providing control signals created locally and autonomously on the medical component for cooperative movement supervision and/or movement control of the quantity of mobile medical components in a facility and/or in a plurality of facility units of the facility.

In accordance with a fourth embodiment, the invention relates to method for operating a mobile medical component of a quantity of mobile medical components in a medical facility comprising a drive unit, a power supply, a communication interface, a sensor unit, a memory unit and a processor unit. The method comprises the following steps:

Activation of the communication interface for continuously receiving navigation signals from each of the mobile medical components of the quantity of mobile medical components in the medical facility;

Receipt of navigation signals from each of the mobile medical components;

Creation of control signals for a drive unit for movement of the mobile medical component based upon the received navigation signals and/or a movement model and/or a request signal;

Creation of navigation signals via a communication protocol based upon the control signals created; and

Sending of the created navigation signals via the communication interface for cooperative supervision and/or control of each mobile medical component of the plurality of mobile medical components in the medical facility and/or in the facility units.

In one form of embodiment of the mobile medical component, this can comprise a user interface for communication with a user. The user interface is embodied to provide visual and/or acoustic information. Moreover the user interface is embodied to receive requests entered by a user.

The inventive form of embodiment of the method described here in accordance with the third embodiment of the invention can also be embodied as a computer program, wherein a computer is made to carry out an embodiment of the inventive method described above when the computer program is executed on a processor of the computer. The computer program can be provided as a signal by download or stored in a memory unit of the computer or of the mobile medical component with computer-readable program code stored therein, to make the mobile medical component execute the instructions in accordance with the method stated above. In this case the computer program can also be stored on a machine-readable storage medium. An alternate solution makes provision for a storage medium that is intended for storage of the computer-implemented method described here and is able to be read by a computer or processor.

The above embodiments and developments, where this makes sense, can be combined with one another in any given way. Further possible embodiments, developments and implementations of the invention also comprise combinations of features of the invention not explicitly stated previously or below with regard to the example embodiments. In particular in such cases the person skilled in the art will also add individual embodiments to the respective basic form of the present invention as an improvement of expansion.

The enclosed drawings are intended to impart a better understanding of the forms of embodiment of the invention. They illustrate forms of embodiment and serve in conjunction with the description to explain principles and concepts of the invention. Other forms of embodiment and many of the advantages stated emerge in respect of the drawings. The elements of the drawings are not necessarily shown true-to-scale.

In the figures of the drawing elements, features and components that are the same, have the same functions and work in the same way—unless stated otherwise—are to be provided with the same reference characters in each case.

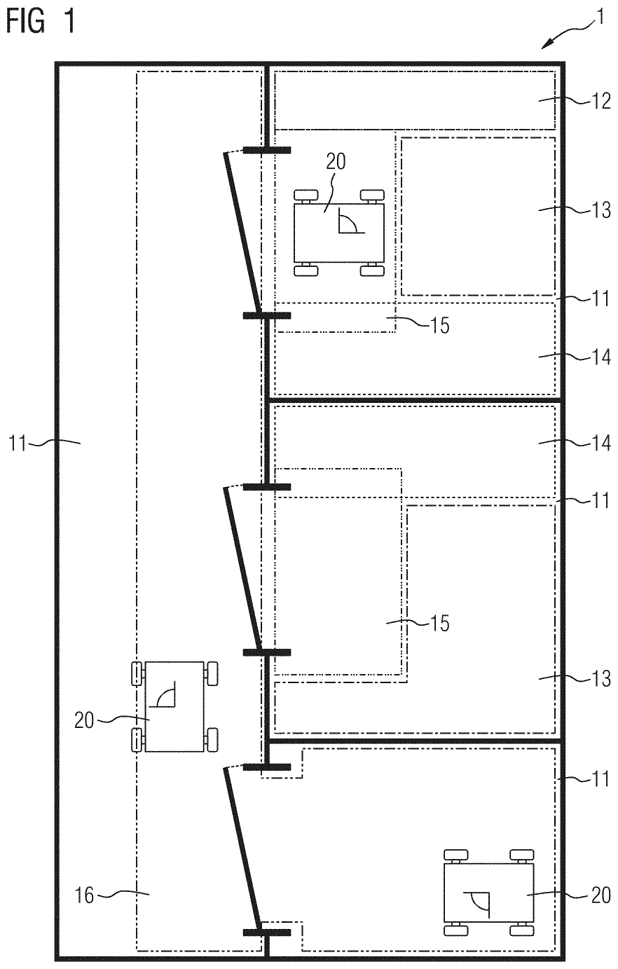

FIG. 1 shows a schematic diagram of a form of embodiment of a system for cooperative movement supervision and/or movement control. The system is implemented in a medical facility 1. The medical facility 1 comprises a hospital, an ambulance, a medical research laboratory, a hospital ward or a polyclinic for example. The medical facility 1 can be divided into a plurality of facility units 11. A facility unit 11 can contain a room, a corridor, a staircase and/or an elevator for example. The facility unit 11 can be divided into a plurality of task-specific zones (12, 13, 14, 15, 16). The task-specific zone can be embodied as a prohibited area 12. No mobile medical component 20 may move through the prohibited area 12.

A plurality of mobile medical components 20 can be used in the medical facility 1. The plurality of mobile medical components 20 communicate with each other via a communication link and communicate continuously via this link, in order cooperatively to monitor and/or to control via a communication protocol the movement of each mobile medical component 20 of the plurality of mobile medical components 20 in the medical facility 1 and/or the facility units 11. A mobile medical component 20 can comprise an examination device for an imaging method, for example CT, MRT, x-ray or ultrasound. Medical components 20 furthermore also comprise the components that are used for recovery and/or life support.

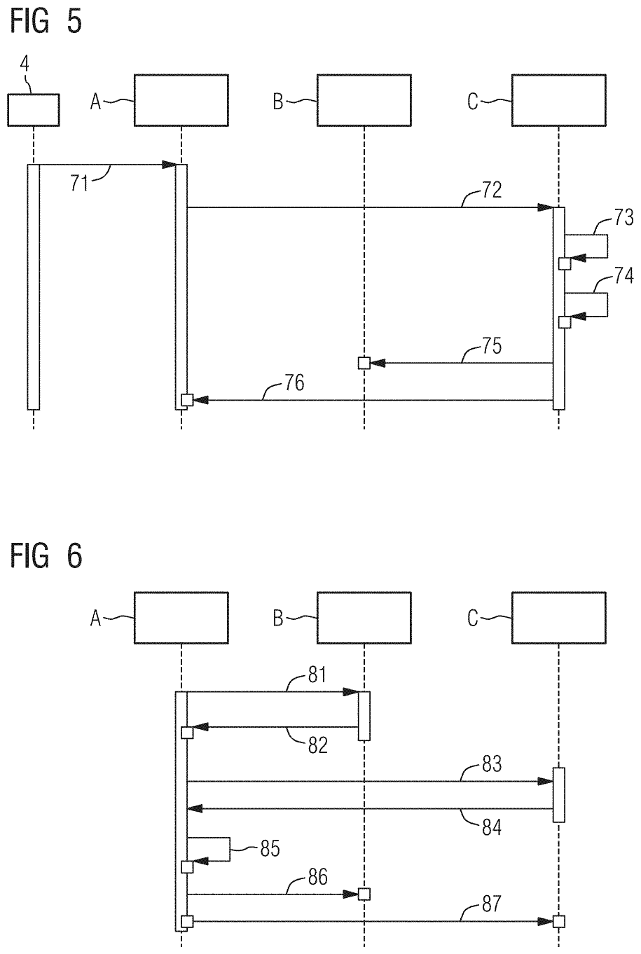

The task-specific zone can furthermore be embodied as a movement area 13. The movement area 13 can be moved through by a mobile medical component 20 after the receipt of an enabling signal. The enabling signal can be provided by a user via a user interface 4 (cf. FIG. 5). Moreover the mobile medical components 20 may only move in the movement area 13 when an enabling signal has been provided for their movement via the user interface 4. In this way the safety can be enhanced for the user in the movement area 13. In one form of embodiment a master-slave principle is applied to the mobile medical components 20 that are positioned in the movement area 13 for the communication and control. Here one mobile medical component 20 is chosen as master and the further mobile medical components 20 are chosen as slaves. The mobile medical component 20 with the master function can remotely control the further mobile medical components 20 in the movement area 13 in an automated manner. The master function of a mobile medical component 20 can also be transmitted to other mobile medical components 20. The transmission can be undertaken by being enabled by a user via the user interface 4. The mobile medical components 20 can also be moved manually by a user in the medical facility 1.

When a mobile medical component 20 cannot move to a destination position in the movement area 13, because for example another mobile medical component 20 is occupying the destination position, there is communication between the mobile medical components 20 involved. The mobile medical component 20 notifies that it would like to move to the destination position. The mobile medical component 20 that is occupying the destination position can decide whether it can leave the movement area 13 and thus the destination position, by moving into the waiting area 14 for example. In one form of embodiment the blocked mobile medical component 20 can show a user via a user interface that a conflict exists. In this case the user can provide a solution to the conflict.

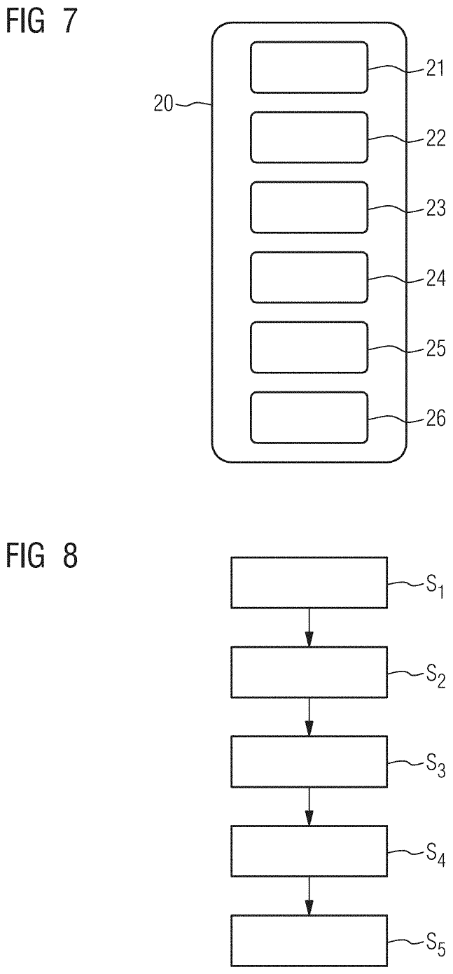

The task-specific zone can furthermore be embodied as a waiting area 14. Mobile medical devices 20 that are not in use can be parked in the waiting area 14. The mobile medical devices 20 can be parked automatically in the waiting area 14 when said devices are no longer needed. For this the mobile medical component 20 is moved into the waiting area 14. There can be an orthogonal departure from the movement area 13 into the waiting area 14. A orthogonal departure can occur for example so that, when enabled by a user, the mobile medical component 20 is moved from the movement area 13 into the transfer area 15 and moves autonomously from the transfer area 15 into the waiting area 14. The transfer area 15 here represents an area via which the transfer between the autonomy area 16 and the movement area 13, as well as the transfer between the movement area 13 and the waiting area 15 is controlled. The waiting area 15 can comprise a plurality of waiting positions. The waiting positions are found automatically. In one form of embodiment the positions of the further mobile medical components 20 are requested for this purpose by mobile medical component 20. Through this it is established which waiting position is still free in the waiting area 15 and can be used. In a further execution position a free waiting position can be established via the sensor unit 24 (cf. FIG. 7) of the mobile medical component 20.

For a movement of a number of mobile medical components 20 the movement of the mobile medical components 20 from the movement area 13 into the waiting area 15 has priority. Only when the mobile medical components 20 that can move from the movement area 13 into the waiting area 15 to a waiting position have fully left the movement area 13 does the movement of another mobile medical component 20 into the movement area 13 take place. The simultaneous inwards and outwards movement of a number of mobile medical components 20 is thus avoided. The collision risk is reduced.

In an advantageous form of embodiment the mobile medical components 20 that are moving on a collision course come to a standstill. The mobile medical components 20 are stopped before a collision occurs. The stop can be signaled to a user via an acoustic and/or visual signal. For example the acoustic and/or visual signal can be provided via a user interface of the mobile medical component 20. The stop can also be transmitted as a stop signal to all or to selected other mobile medical components.

The autonomy area 16 represents a zone in which the mobile medical component 20 moves autonomously without user specifications. The autonomy area 16 comprises the floors, passages and corridors, as well as stores for parking the mobile medical components 20. If a new mobile medical component 20 is needed in a specific movement area 13 of a facility unit 11, this can be requested via a request signal. The request signal can be provided via the interface 4. The requested mobile medical component 20 then moves autonomously, for example from a store into the autonomy area 16. Via the autonomy area 16 the mobile medical component 20 can move itself independently as far as the transfer area 15 of the respective facility unit 11. The reaching of the transfer area 15 of the respective facility unit 11 is signaled to a user, for example via a display in the user interface 4. An enabling signal must be actively provided via the user interface 4 before the requested mobile medical component 20 can move into the movement area 13 of the facility unit 11. In a preferred form of embodiment the requested mobile medical component 20 also notifies the master component that is currently active. In one form of embodiment the autonomy area 16 is designed so that a lane is provided for each direction of movement. In a further form of embodiment the lanes are embodied with an autonomous lane guidance. The lane guidance can also be learned via a predetermined movement profile.

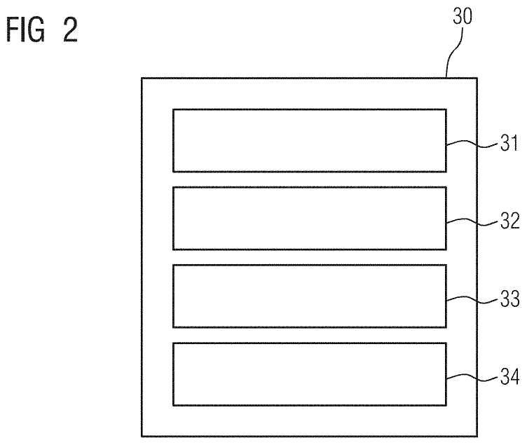

FIG. 2 shows a block diagram of a communication layer model 30 for communication between the mobile medical components 20.

The layer 31 comprises the cooperative communication protocol, for example a movement planner protocol in accordance with an embodiment of the present invention.

The following messages are defined by the cooperative movement planner protocol:

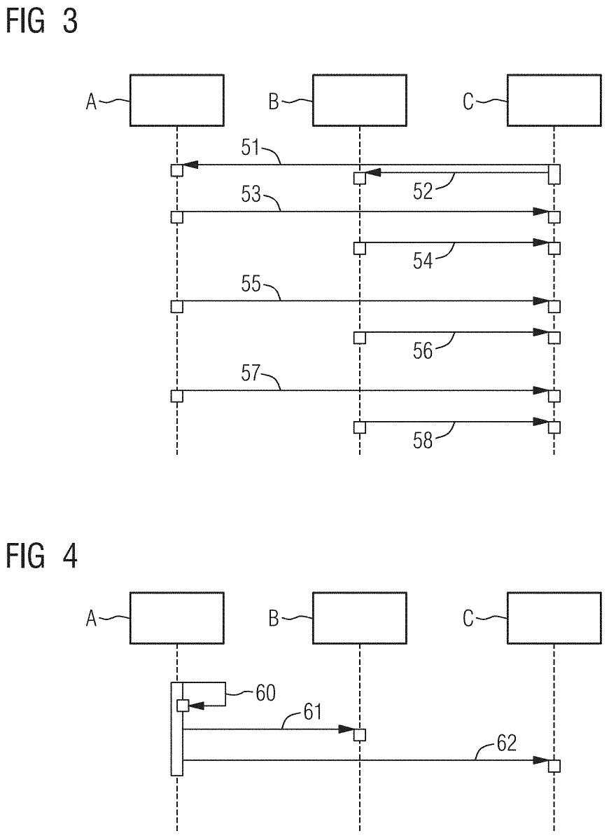

/robot_broadcast 51 (cf. FIG. 3) comprises a broadcast message for registering a new mobile medical component 20;

/robot_status 53, 54, 75, 76 (cf. FIG. 3) comprises a status message, which sends the current status of the mobile medical component 20, such as for example the name, the status, whether the mobile medical component 20 is in the master or slave function;

/map_update 55, 56, 61, 62 (cf. FIG. 3) comprises the sending of an update of the map to all mobile medical components 20; A occupancy grid similar to that of the Robot Operating System (ROS) can be used, which breaks down the available space based upon cells and stores for each cell whether the cell is occupied or not;

/floor_update 57, 58, 86, 87 (cf. FIG. 3) comprises an update of the floor plan with movement areas in the facility units 11; moreover for each facility unit 11 the standard master and connection data is stored;

/move_request 72 (cf. 72) comprises the request for a movement of a mobile medical component 20 to a destination position on the map in accordance with the rules set down above;

/master request 81, 83 (cf. FIG. 6) comprises the request for the transfer of the master function from another mobile medical component 20.

The layer 32 comprises the Robot Operating Messaging system (ROS). Via the Robot Operating Messaging system, in an advantageous manner, there can be a distributed communication via a number of mobile medical components 20 without a dedicated communication master.

The layer 33 comprises the transport layer. In the transport layer there is the segmentation of the flow of data and the avoidance of congestion. Via the transport layer a unified access to the application-oriented layers 31, 32 is provided, so that the characteristics of the communication network are not to be taken into consideration. TCP/IP (Transmission Control Protocol/Internet Protocol), UDP (User Datagram Protocol), SCTP (Stream Control Transmission Protocol), DCCP (Datagram Congestion Control Protocol) can be used as transmission protocols.

The layer 34 comprises the data link layer, which is intended to guarantee a reliable and error-free transmission and is intended to provide the access to the transmission medium, for example WLAN, Bluetooth, Bluetooth Low Energy.

Via the communication protocol any given number of mobile medical components 20 can be added, supervised and controlled. Moreover no central planning and/or cooperation computer is needed. The supervision and/or control is done locally at the respective mobile medical components 20.

FIG. 3 shows a schematic diagram of a timing sequence of the registration of a new mobile medical component 20. In FIG. 3 the reference characters A, B and C refer to a first, second and third mobile medical component 20. Shown in FIG. 3 is the registration of a new mobile medical component C. The new mobile medical component C sends a/robot_broadcast message 51, 52 to the available mobile medical components A, B and makes itself known. The available medical components A, B, as a response to the/robot_broadcast message 51, 52, send their status via the/robot_status message 53, 54 to the new mobile medical component C. The available medical components A, B moreover send a map and a plan of the facility units 11 via the/map_update message 55, 56 and via the/floor_update message 57, 58. The new mobile medical component C can create a map and a plan of the facility units 11 from the data.

FIG. 4 shows a schematic diagram of a time sequence of the data communication between the mobile medical components A, B, C. The data communication can comprise data for updating a map for example. In FIG. 4 the first mobile medical unit A has detected new information via the sensor unit 24 (cf. FIG. 7) and updates the map 60 based upon the new information. The first mobile medical unit A sends the/map_update message 61, 61 to the further mobile medical units B, C. The maps of the further mobile medical units B, C are updated.

FIG. 5 shows a schematic diagram of a time sequence of the movement request of a mobile medical component A, B, C. In FIG. 5 for example a new mobile medical component C is requested from a store. For the request a request signal is provided via the user interface 4. The request signal can comprise the information about the requested mobile medical component and the requested movement area 13 in a facility unit 11. The third mobile medical component C receives a /move_request message 72. The third mobile medical component C checks 73 whether the transfer area 15 of the requested facility unit 11 is free. If the result of the check 73 is that the transfer area 15 of the requesting facility unit 11 is free, an autonomous movement of the third mobile medical component C is started 74. During the movement the third mobile medical component C transfers its status, for example the position to the further mobile medical units A, B using the/robot_status message 75, 76. This is the case if the further mobile medical devices A, B are (present) in the requesting facility unit 11 and the user interface of the first mobile medical component A has been used for the request of the third mobile medical component.

FIG. 6 shows a schematic diagram of a time sequence of a master request of a mobile medical component 20. In the example shown in FIG. 6, the second mobile medical component B is set as master and the further mobile medical components A, C are slaves. The first mobile medical component A sends a /master request message 81, 83 to the mobile medical components B, C, in order to bring about a switch to ‘master’. The mobile medical components B, C confirm 82, 84 the receipt of the request and the second mobile medical component B as the current master acknowledges the master function. The first mobile medical component A sets 85 the master function. As the master component, the first mobile medical component A can start an update of the floor plan via the/floor_update message 86, 87.

FIG. 7 shows a further block diagram in accordance with a form of embodiment of the mobile medical components 20. The mobile medical component 20 from a quantity of mobile medical components 20 for cooperative movement supervision and/or movement control of the quantity of mobile medical components 20 in a medical facility 1 comprises a drive unit 21. The drive unit 21 is embodied for driving the mobile medical component 20. The drive unit is embodied as a DC motor. The mobile medical component 20 furthermore comprises a power supply 22. The power supply is embodied for supplying electrical power to the individual components of the mobile medical component 20. The power supply 22 comprises a DC power supply, which can be charged from a DC power source or an AC power source with an AC/DC converter. The charging can take place in an advantageous manner during a waiting period in a waiting area 15 or in a store.

Moreover the mobile medical component 20 comprises a communication interface 23. The communication interface 23 is embodied for setting up a communication link with further mobile medical components 20 in a medical facility 1. The communication interface is embodied to set up a WLAN connection, Bluetooth or Bluetooth low Energy connection.

Furthermore the mobile medical component 20 comprises a sensor unit 24 for detecting the environment of the mobile medical component 20. The sensor unit 24 is embodied to scan the environment of the mobile medical component 20 and to provide information about its distance from and the embodiment of objects within the environment. The sensor unit 24 comprises lidar, radar, laser and/or a camera for example. Based upon the information provided a map can be created. The map can be transmitted to the further mobile medical components 20. Moreover the mobile medical component 20 comprises a memory unit 25 for storage of the learned movement model and/or of maps of the medical facility 1. Moreover the mobile medical component 20 comprises a processor unit 26 for cooperative supervision and/or control of the movement of each mobile medical component 20 in the medical facility 1 and/or in the facility units 11 by provision of control signals to activate the drive unit 21.

In a preferred form of embodiment of the invention, the mobile medical components 20 can be embodied with different resources, so that e.g. a first mobile medical component 20 is embodied with few processing resources and a second mobile medical component 20 with relatively high processing resources by comparison. The first mobile medical component 20 can get the second mobile medical component 20 to take over its computations (e.g. for navigation, for constructing the map and/or for computing as part of the control and supervision) temporarily or as its representative and to send the result to the first mobile medical component 20. The first mobile medical component 20 can thus be freed from the computing load but still take part in the method.

In one form of embodiment, the mobile medical component 20 comprises a user interface. The user interface can comprise an input unit, for example an input panel for receiving user inputs, and an output unit, for example a display for output of information. Furthermore the user interface can comprise an acoustic output unit, for example a loudspeaker. In an alternate form of embodiment the user interface can be integrated entirely into a touch display or a handheld device and comprise a loudspeaker for outputting acoustic warning tones.

FIG. 8 shows a flow diagram in accordance with one form of embodiment of the inventive method. In the form of embodiment shown the method comprises a number of steps. In a first step S1 the communication interface 23 is activated for continuous receipt of navigation signals from each of the mobile medical components 20 of the quantity of mobile medical components 20 in the medical facility 1. In a further step S2 there is a receipt of navigation signals from each of the mobile medical components 20. In a further step S3 control signals for the drive unit for movement of the mobile medical component 20 based upon the navigation signals received and/or of a movement model and/or of a request signal are created. In one form of embodiment the movement model can be learned via a manually driven movement profile.

In a further form of embodiment the movement model can be learned via a predetermined movement profile. In a further step S4 navigation signals are created via a communication protocol based upon the control signals created. The messages for navigation of the mobile medical components 20 in the medical facility 1 are defined via the communication protocol. In a further step S5 the navigation signals created are sent via the communication interface 23 for cooperative supervision and/or control of each mobile medical component 20 of the plurality of mobile medical components 20 in the medical facility 1 and/or in the facility units 11.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A system for at least one of cooperative movement supervision and movement control of mobile medical components in a medical facility including a plurality of facility units, comprising:
a plurality of mobile medical components, to exchange data via a communication link and communicate continuously, and to, via a communication protocol, at least one of cooperatively supervise and control the movement of each of the mobile medical components of the plurality of mobile medical components, at least one of in the medical facility and in the plurality of facility units.

2. The system of claim 1, wherein the communication protocol of the mobile medical components is embodied as a master-slave protocol, wherein one mobile medical component, of the plurality of mobile medical components, is chosen as the master and the further mobile medical components, of the plurality of mobile medical components, are chosen as slaves.

3. The system of claim 1, wherein a facility unit, of the plurality of facility units of the medical facility, includes at least two separate task-specific zones.

4. The system of claim 3, wherein one task-specific zone, of the at least two separate task-specific zones, includes a prohibited area, a transfer area, a movement area, a waiting area or an autonomy area.

5. The system of claim 4, wherein the movement of a mobile medical component into the movement area is enabled via an enabling signal.

6. The system of claim 1, wherein the mobile medical components are configured to move autonomously in the medical facility, via a learned movement model of the medical facility.

7. The system of claim 6, wherein the movement model is learned via a manually driven movement profile.

8. The system of claim 6, wherein the movement is learned via a predetermined movement profile.

9. The system of claim 1, wherein the mobile medical components are oriented and are configured to move autonomously in the medical facility, via a localization system.

10. The system of claim 1, wherein the mobile medical components are configured to move autonomously in the medical facility via a SLAM method.

11. A mobile medical component including a quantity of mobile medical components for at least one of cooperative movement supervision and cooperative movement control of the quantity of mobile medical components, comprising:
a driver, embodied to drive the mobile medical component;
a power supply, embodied to supply electrical power to components of the mobile medical component;
a communication interface, embodied to set up a communication link to further mobile medical components in a medical facility;
a sensor to detect an environment of the mobile medical component;
a memory to store at least one of a learned movement model and maps of the medical facility; and
a processor for at least one of cooperative supervision and cooperative control of movement of each mobile medical component in at least one of the medical facility and facility units, through provision of control signals for activating the drive unit.

12. A method for operating a system including a plurality of mobile medical components, to exchange data via a communication link and communicate continuously, and to, via a communication protocol, at least one of cooperatively supervise and control the movement of each of the mobile medical components of the plurality of mobile medical components, at least one of in the medical facility and in the plurality of facility units, comprising:
provisioning control signals created locally and autonomously on the medical component for at least one of cooperative movement supervision and cooperative movement control of the quantity of mobile medical components in at least one of a facility and a plurality of facility units of the facility.

13. A method for operating a mobile medical component of a quantity of mobile medical components in a medical facility including a driver, a power supply, a communication interface, a sensor, a memory and a processor, the method comprising:
activating the communication interface for continuous receipt of navigation signals from each of the mobile medical components of the quantity of mobile medical components in the medical facility;
receiving navigation signals from each of the mobile medical components;
creating control signals for the driver for movement of the mobile medical component based upon at least one of the navigation signals received, a movement model and a request signal;
creating navigation signals via a communication protocol based upon the control signals created; and
sending the navigation signals created, via the communication interface, for at least one of cooperative supervision and cooperative controlling each mobile medical component of the plurality of mobile medical components in at least one of the medical facility and the facility units.

14. A non-transitory computer readable medium storing a computer program including program code for carrying out the method of claim 13 when the computer program is executed on an electronic device.

15. The system of claim 2, wherein a facility unit, of the plurality of facility units of the medical facility, includes at least two separate task-specific zones.

16. The system of claim 2, wherein the mobile medical components are configured to move autonomously in the medical facility, via a learned movement model of the medical facility.

17. The system of claim 2, wherein the mobile medical components are oriented and are configured to move autonomously in the medical facility, via a localization system.

18. The system of claim 2, wherein the mobile medical components are configured to move autonomously in the medical facility via a SLAM method.
